(19) United States
(12) Patent Application Publication
(10) Pub. No.: US 2011/0108026 A1
(43) Pub. Date: May 12, 2011

(54) INHALER

(76) Inventor: Mark Boardman, Grange (AU)

(21) Appl. No.: 12/917,299

(22) Filed: Nov. 1, 2010

Related U.S. Application Data
(60) Provisional application No. 61/259,750, filed on Nov. 10, 2009.

(51) Int. Cl. A61M 11/00

(52) U.S. Cl. 128/200.23

(57) ABSTRACT

An inhaler having a housing which defines a receptacle shaped and dimensioned for receiving a pressurised medicament canister having a nozzle, and a mouthpiece arranged in fluid communication with the receptacle by means of a conduit and an actuator including a button fast with a duct which is shaped and configured for fitment into the conduit, the duct configured to arrange the mouthpiece and nozzle in fluid communication when the button is pressed, thereby actuating the canister.

### CROSS REFERENCE TO RELATED APPLICATIONS

This application claims the benefit under 35 U.S.C. 119 of U.S. Patent Application No. 61/259,750 filed Nov. 10, 2009, titled “WATERTIGHT INHALER”.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

This invention relates to an inhaler used for the administration of inhalants, which in one example can be used in an aquatic environment.

### DESCRIPTION OF THE PRIOR ART

Reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

U.S. Pat. No. 5,855,307 provides for an inhaler or container holster which is strapped around a user's wrist and ready to use. End hook and look fasteners on a wrist band hold an inhaler band to the user's wrist band while a second set of hook and loop end fasteners on an inhaler band hold the inhaler. The wrist band is generally perpendicular to the inhaler band which either totally or partially encircles and holds the canister containing the inhaler. The canister's end segments may be additionally secured to the inhaler band by elastic strap assemblies attached to the inhaler band which loop around the top and bottom of the canister at opposite ends. Ventolin or any other asthma medication may be placed in the inhaler or container.

GB 2,363,991 teaches a carrier for an inhaler attachable to clothing made of durable waterproof flexible material which holds the inhaler and which is attached to clothing by spring clip means attached to the carrier. The flexible material may form a pocket and the carrier may have a flap protecting the inhaler.

AU 2003275808 discloses breathing apparatus for medicating an airstream, comprising a breathing apparatus, a medication chamber adapted to store and discharge a therapeutic agent, a delivery pathway between the chamber and an intake air pathway and releasing means for selectively discharging the therapeutic agent from the chamber into the intake air pathway. The apparatus is particularly suited for scuba diving, snorkels, gas masks and filters. The invention is suitable for asthma sufferers but also extends to other respiratory diseases. Also described are a medication chamber for use in medicating an airstream and a method of medicating an airstream.

AU 2004273547 describes a medication holder comprising a housing having an outer wall and an inner wall preferably in sleeved relationship to each other. The outer wall may be moved between a closed position in which the housing is sealed for ingress of moisture or other contaminants and an open position providing access to the medication container discharge outlet. The second wall may be slid longitudinally or rotated circumferentially to access the cavity and may, in the process, open an air pathway through the cavity. The medication container is preferably a pressurised canister. Alternatively, it may comprise a frangible vial with a volatile liquid. In the latter case, an evaporation grid may also be supplied to enhance absorption of the liquid which is preferably methoxyfluorane. Opening means may be provided to open the frangible vial and discharge the contents onto the evaporation means which is in fluid contact with the air pathway. A medication chute may be rotatable between stowed and deployed positions. The chute may include an auxiliary air inlet. The medication holder may also include one-way valves to channel inspiratory air through the chamber and direct expiratory air elsewhere. The expiratory air may pass through unidirectional valve and also through an absorbent filter to minimise environmental contamination.

AU 2006225241 provides a portable medication holder for discharging liquid agent into an air stream. The holder comprises a housing having a first wall and a second wall with the walls movable relative to each other. The second wall may be moved between a closed position and an open position wherein an air pathway is open. The holder has evaporation means to assist in evaporation of the liquid agent into air in the air pathway which flows through a medication discharge chute to a user. The device also include opening means for opening the medication container and releasing the liquid agent to the air pathway and one or more one-way valves to provide uni-directional air flow for one or both of the inspiratory air and expired air. The liquid agent is preferably methoxyflurane. The device may include an auxiliary air inlet for varying the concentration of inhaled agent. The holder is preferably sealed against the external environment. The holder may include a lanyard adapted to activate relative movement between the walls. It may also include a rip seal opening for one end and a deformable cap which may be sprung clear of the medication discharge chute by impact against a surface.

AU 2006284521 describes a medication dispenser for receiving a pressurised medication container and discharging therapeutic doses therefrom. The medication dispenser comprises an elongate tubular body with a cavity formed in the body and adapted to receive a medication container. A lateral aperture is formed in the body and communicates with the cavity. A sleeve is rotatably mounted to a first end region of the medication dispenser. The sleeve has a sleeve aperture which moves into and out of alignment with the lateral aperture during rotation of the sleeve. A mouthpiece moves between a discharge position and a storage position with rotation of the sleeve. The tubular body has a longitudinal axis which deviates away from the position of the extended mouthpiece, the deviation occurring outside the first end region. The invention may extend to a carrier for use with the medication dispenser.

None of the known prior art teaches an inhaler which can be safely and conveniently used in an aquatic environment, such as by a surfer, a swimmer, or the like. The prior art inhalers do not provide for adequate sealing to prevent the ingress of water when these inhalers are used in an aquatic environment. This affects the ability of the prior art inhalers to effectively deliver medicament, as the ingress of water can lead to blockages and other related malfunctions.

### SUMMARY OF THE PRESENT INVENTION

According to one aspect of the invention there is provided an inhaler having:

a housing which defines a receptacle shaped and dimensioned for receiving a pressurised medicament canister having a nozzle, and a mouthpiece arranged in fluid communication with the receptacle by means of a conduit; and,

an actuator including a button fast with a duct which is shaped and configured for fitment into the conduit, the duct configured to arrange the mouthpiece and nozzle in fluid communication when the button is pressed, thereby actuating the canister.

Typically the inhaler includes a sealing cap displaceable between a closed position, in which the cap engages the housing to at least one of seal and lock the button and seal the mouthpiece, and an open position, in which the cap is disengaged from the housing to expose the mouthpiece and the button.

Typically the inhaler includes an attachment mechanism shaped and configured for removably attaching the housing to a body of a person.

Typically, the housing includes an elongate tubular structure with a first end forming an entry to the receptacle with the mouthpiece defined on a side of the housing proximate a second end.

Typically, the housing defines the conduit substantially coaxial with the elongate tubular housing so that said conduit extends from the receptacle to the second end of the housing.

Typically, the duct extends transversely from the button so that the button lies substantially coplanar with the second end of the housing when the duct is fitted inside the conduit.

Typically, the second end of the housing is shaped and configured to receive the button so that said button lies flush with the second end of the housing.

Typically, the sealing cap engages the second end of the housing when in the closed position.

Typically, the attachment mechanism includes a receptacle cap configured to engage the first end of the housing to seal the canister inside the receptacle in a fluid tight manner.

Typically, the duct includes at least one O-ring seal to seal the duct inside the conduit in a fluid tight manner.

Typically, the duct is configured to actuate the nozzle of the canister when the button is pressed to provide a passageway for the medicament expelled from the nozzle through the duct to the mouthpiece.

Typically, the sealing cap is configured to seal the button and mouthpiece in a fluid tight manner when in the closed position.

Typically, the mouthpiece includes air intake apertures to facilitate a person operatively inhaling the medicament.

Typically, the sealing cap covers and seals the air inlet apertures when in the closed position.

Typically, the sealing cap includes a tether to secure the sealing cap to the housing when in the open position.

Typically, the sealing cap defines a hinge portion which enables the cap to split into a front portion and a rear portion in the open position.

Typically, the attachment mechanism includes an elastic strap for attaching the housing to a limb of the person's body.

Typically, the strap is configured to attach the housing to a forearm of the person such that the housing lies substantially parallel with the person's forearm.

Typically, the attachment mechanism operatively holds the canister immobile inside the receptacle.

Typically, the attachment mechanism is configured to operatively seal the canister inside the receptacle.

Typically, the inhaler includes a pressurised medicament canister.

Typically, the mouthpiece defines a mouthpiece aperture via which the duct arranges the mouthpiece in fluid communication with the nozzle when the button is pressed.

Typically, the mouthpiece aperture has a larger diameter than an aperture in which the duct terminates.

### BRIEF DESCRIPTION OF THE DRAWINGS

An example of the present invention will now be described with reference to the accompanying drawings, in which:—

FIG. 1 shows a perspective representation of an example of an inhaler with a sealing cap in a closed position;

FIG. 2 shows a top view of the inhaler of FIG. 1;

FIG. 3 shows a side view of the inhaler of FIG. 1;

FIG. 4 shows a front view of the inhaler of FIG. 1;

FIG. 5 shows a perspective representation of the inhaler of FIG. 1 with a sealing cap in an open position;

FIG. 6 shows a top view of the inhaler of FIG. 5;

FIG. 7 shows a side view of the inhaler of FIG. 5;

FIG. 8 shows a front view of the inhaler of FIG. 5;

FIG. 9 shows an exploded view of the inhaler of FIGS. 1 and 5;

FIGS. 10A and 10B show a cross-sectional side view along line A-A of the inhaler of FIGS. 1 and 5 with the sealing cap in the closed position;

FIGS. 11A and 11B show a cross-sectional side view along line B-B of the inhaler of FIGS. 1 and 5 with the sealing cap in the open position and an actuator in an operative position;

FIGS. 12A and 12B show an example of a manner in which the actuator of the inhaler is inserted into the inhaler;

FIGS. 13A and 13B show another example of the inhaler having a receptacle cap;

FIGS. 14A and 14B show a further example of a sealing cap for the inhaler; and

FIGS. 15A to 15E show a further example of an attachment mechanism of the inhaler.

### DETAILED DESCRIPTION OF THE PREFERRED EMBODIMENTS

With reference now to the accompanying drawings, there is shown one example of an inhaler 10 typically used for the inhalation of asthma reliever medication, such as Ventolin or the like. However, it is to be appreciated that that inhaler 10 may be used for any type of inhalant or similar medicament.

One example of the inhaler 10 includes a housing 12, an actuator 14, a sealing cap 16 and some manner of attachment mechanism 18 for removably attaching the housing to a body of a person. The inhaler 10 includes a number of features, as described in more detail below, that enable the inhaler 10 to find particular application for use in aquatic environments, such as the ocean. An important aspect of the inhaler 10 is to maintain a pressurised medicament canister 22 in a watertight manner, as well as properly seal a delivery system of the inhaler 10 against the ingress of liquid until placed in an operative condition prior to use thereof. The respective components of the inhaler 10 are generally manufactured from suitable polymers, such as thermoplastic elastomers, polypropylene, and the like, that are suitable for use in an aquatic environment.

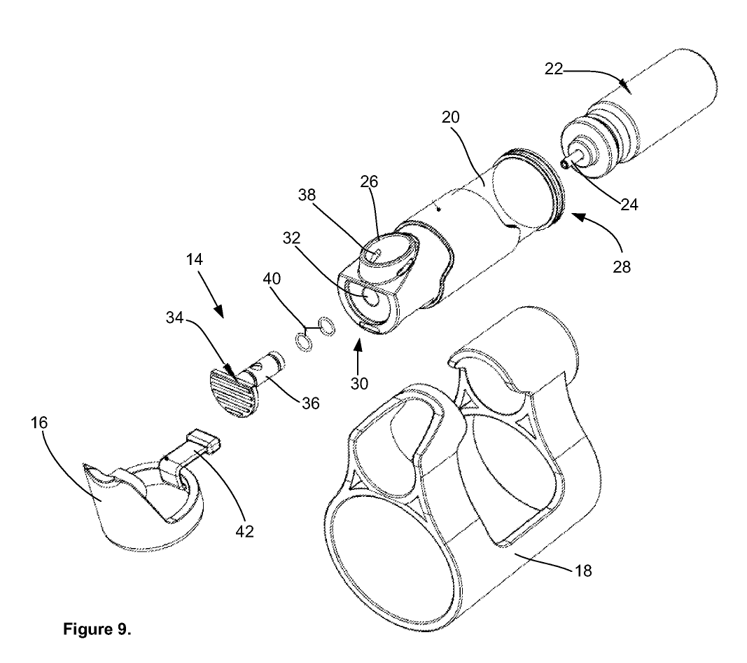

The housing 12 generally defines a receptacle 20 which is shaped and dimensioned for receiving the pressurised medicament canister 22 having a nozzle 24. The housing 12 also defines a mouthpiece 26 which is arranged in fluid communication with the receptacle 20 by means of a conduit 32. In one example, the housing 12 typically includes an elongate tubular structure with a first end 28 forming an entry to the receptacle 20 with the mouthpiece 26 defined on a side of the housing 12 proximate a second end 30, as shown in more detail in FIG. 9.

In use, the canister 22 is placed through the first end 28 into the receptacle 22 where it fits snugly into the housing 12. The attachment mechanism 18 is configured to operatively seal the canister 22 inside the receptacle 20. In one example, the attachment mechanism 12 includes an elastic strap for attaching the housing to a limb of the person's body, or the like. The strap is typically configured to attach the housing 12 to a forearm of the person such that the housing 12 lies substantially parallel with the person's forearm. This configuration facilitates in ergonomic wearing and use of the inhaler 10.

In one example, the attachment mechanism 12 operatively holds the canister 22 immobile inside the receptacle 20. As can be seen in FIGS. 9, 10 and 11, the first end 28 of the housing 12 typically includes ridges 44 shaped and dimensioned to complementarily engage suitable slots of the attachment mechanism so that the canister 22 is held tightly in the receptacle 20. The attachment mechanism 18 also includes a retaining protrusion to further secure the canister inside the receptacle 20 such that the canister cannot move. It will be appreciated however that alternative attachment mechanisms may be used, such as friction fit, clip fit or the like.

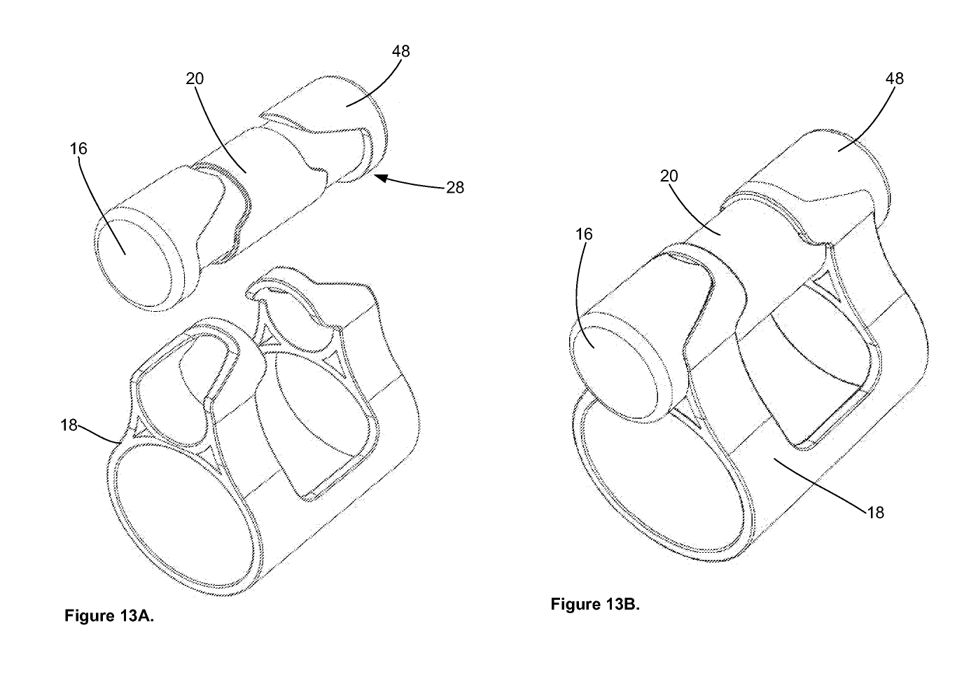

Alternatively, in another example shown in FIGS. 13A and 13B, the attachment mechanism 18 includes a receptacle cap 48 configured to engage the first end 28 of the housing 12 to seal the canister 22 inside the receptacle 20 in a watertight manner. In the shown example, the attachment mechanism 18 does not fit over the receptacle cap 48, but in further examples this need not be the case and the attachment mechanism 18 may enclose the receptacle cap 48.

The attachment mechanism or strap 18 of the current example is typically manufactured from a suitable polymer to allow a person to fit the strap over their hand to sit on their forearm. Alternatively, in other examples the attachment mechanism 18 may form part of a wetsuit, or the like. It is to be appreciated that the attachment mechanism 18 may include any suitable means for removable attaching the housing to a person's body.

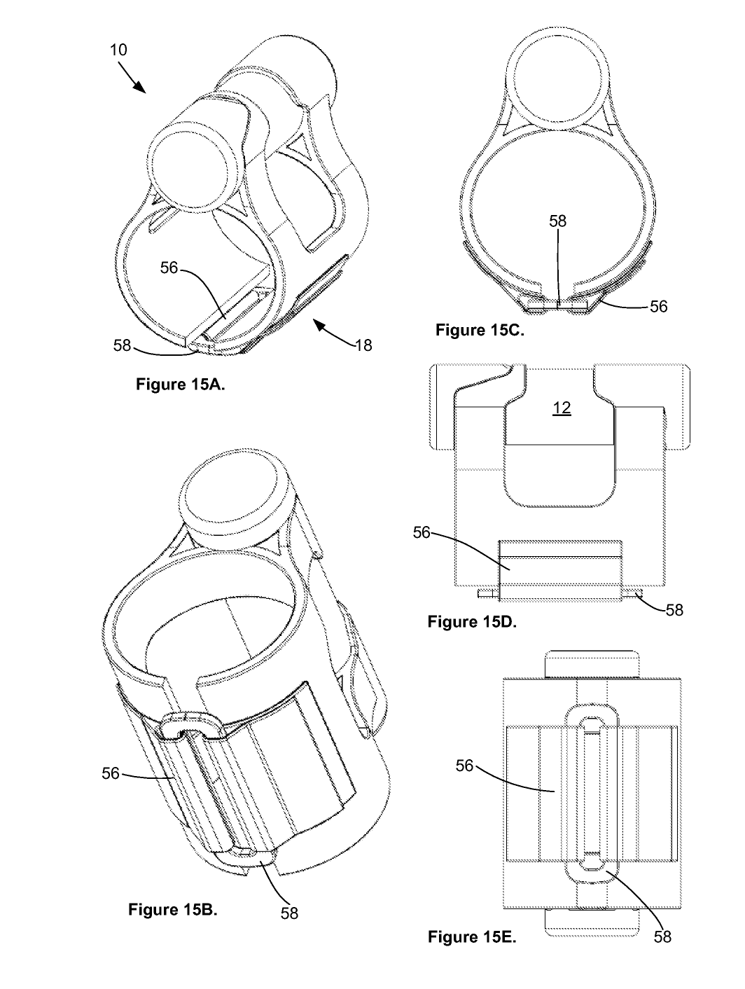

For example, FIG. 15 show another example of the attachment mechanism 18. In this example, the attachment mechanism 18 includes a strap 56 and a buckle 58, as shown, which allows the attachment mechanism to be adjustable to fit different users. The strap 56 is typically manufactured from Neoprene™ or a similar fabric. The strap 56 includes a slit at the bottom, as shown, with the buckle 58 fastened to the strap 56. The buckle 58 may include a press-fit connection to secure the inhaler to a user's arm. Alternatively, the strap may include hook and loop fasteners at suitable locations to fix the inhaler 10 to a user's arm. The strap 56 is typically made from a comfortable material or fabric which provides breathability. This would also allow the inhaler to be comfortably worn outside an aquatic environment.

As mentioned above, the housing 12 also defines a mouthpiece 26 on a side thereof proximate the second end 30. During use, a user will place his/her mouth over the mouthpiece in order to inhale the medicament from the canister 22. It is to be appreciated that proper inhalation of medicament from the canister 22 is generally facilitated when air is mixed with such medicament during inhalation. As such, the mouthpiece 26 generally includes air intake apertures 38, as shown, to facilitate a person operatively inhaling the medicament.

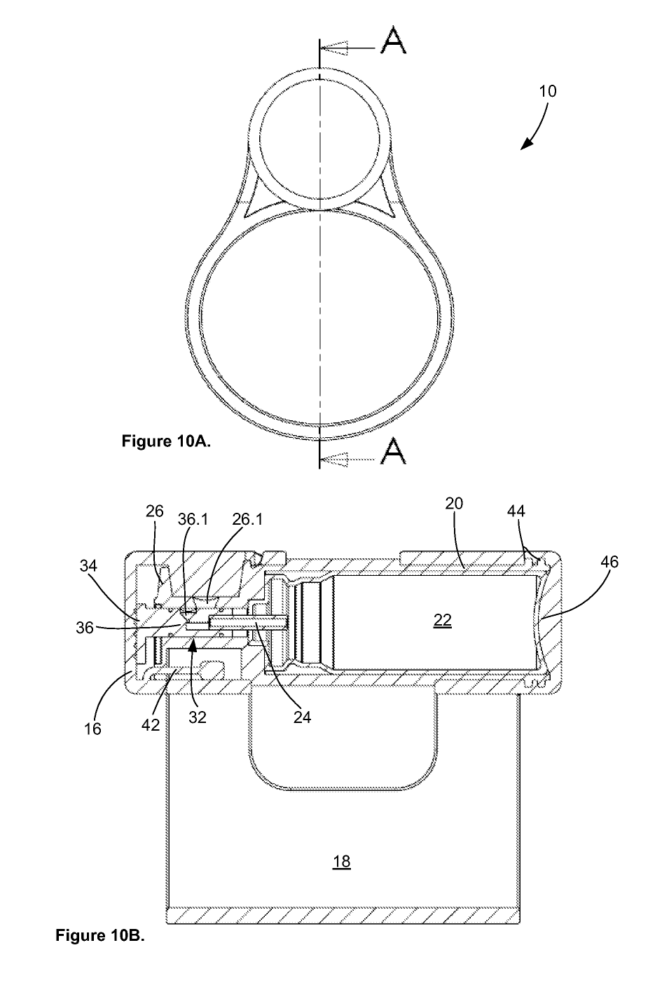

The housing 12 further includes a conduit 32 arranging the mouthpiece 26 in fluid communication with the receptacle 20. The nozzle 24 of the canister 22 is typically placed into the conduit 32, as shown in FIGS. 10 and 11. In the current example, the housing 12 defines the conduit 32 such that it lies substantially coaxial with the elongate tubular housing 12 so that the conduit 32 extends from the receptacle 22 to the second end of the housing 30.

The inhaler 10 also includes an actuator 14 for operatively actuating the nozzle 24 of the medicament canister 22. The actuator 14 includes a button 34 which is fast with a duct 36. The duct 36 is shaped and configured for fitment into the conduit 32 of the housing 12. The duct 36 is configured to operatively arrange the mouthpiece 26 and nozzle 24 of the canister 22 in fluid communication when the button 34 is pressed.

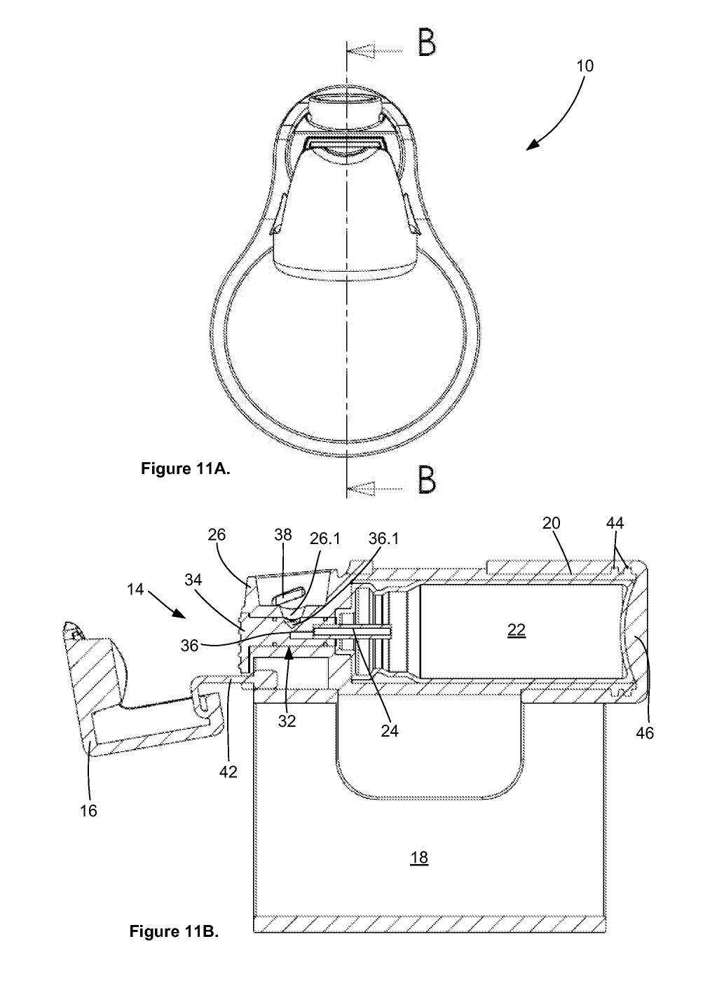

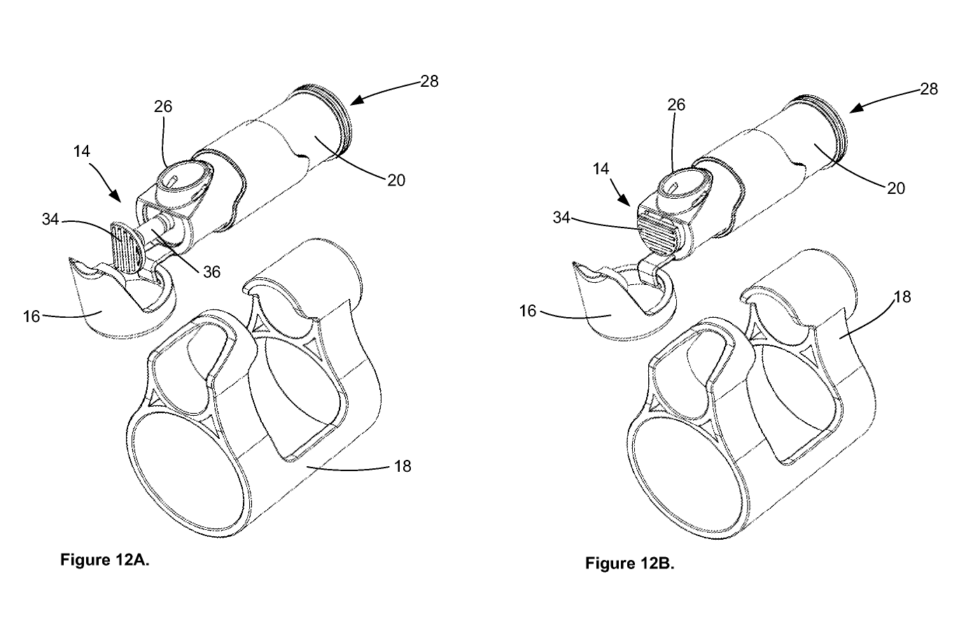

The duct 36 fits snugly inside the conduit 32 and includes two O-ring seals 40 to seal the duct 36 inside the conduit 32 to prevent water from reaching the canister 22, as well as to prevent medicament from escaping from the inhaler 10. FIGS. 12A and 12B show an example of how the duct 36 of the actuator 14 is located inside the conduit 32 of the housing 12. The duct 36 and conduit 32 typically include complementary fittings (not shown) so that the duct 36 must be turned when inserted into the conduit 32, as shown, to engage the conduit 32 to ensure that the duct is secured in place as well as in proper alignment with the mouthpiece 26. The fittings may also facilitate proper fluid tight sealing in conjunction with the O-rings 40. As mentioned above, the duct 36 is configured to actuate the nozzle 24 of the canister 22 when the button 34 is pressed to provide a passageway for the medicament expelled from the nozzle 24 through the duct 36 to the mouthpiece 26.

To perform this function, the duct 36 extends transversely from the button 34 so that the button 34 lies substantially coplanar with the second end 30 of the housing 12 when the duct 36 is fitted inside the conduit 32. The second end 30 of the housing 12 is also typically shaped and configured to receive the button 34 so that the button 34 lies flush with the second end 30 of the housing 12 when the button 34 is pressed, as shown in FIG. 11B.

The operation of the duct 36 can be more clearly seen in FIGS. 10 and 11. FIG. 10 shows the button 34 when not pressed. As can be seen, the duct 36 is out of alignment with the mouthpiece 26 and the nozzle 24 is not actuated. Specifically, the duct 36 terminates in duct aperture 36.1, as shown. Similarly, the mouthpiece 26 defines a mouthpiece aperture 26.1, as shown. When the button 34 is not depressed, the duct aperture 36.1 and the mouthpiece aperture 26.1 do not align, as shown in FIG. 10.

FIG. 11 shows the button 34 when depressed, where the duct 36 is pushed down the conduit 32 to actuate the nozzle 24 and the duct 36 is simultaneously pushed into alignment with the mouthpiece 26. As a result, the duct aperture 36.1 aligns with the mouthpiece aperture 26.1 so that any medicament expelled from the canister 22 can be inhaled via the mouthpiece 26. This provides a fluid passageway through the duct for medicament expelled from the nozzle 24 to travel to the mouthpiece 26 for inhalation. It is to be appreciated that during the pressing of the button 34, the canister 22 does not move inside the receptacle 20, but rather that the duct 36 is displaced to actuate the nozzle 24. In the current example, the canister 22 is securely retained in the receptacle 20 by means of the attachment mechanism 18.

Maintaining the canister 22 stationary during use, by allowing activation of the inhaler using a moving button 34, leads to a number of benefits over traditional inhaler arrangements in which activation is achieved by movement of a canister. For example, the arrangement allows all moving parts to be mechanically isolated at one of the housing 12. This tends to make manufacturing easier and hence cheaper than traditional arrangements, as well as making the inhaler easier to hold and actuate. The arrangement also makes the inhaler easier to seal, for example by allowing the canister to be retained within the housing 12 by the receptacle cap 48, thereby preventing ingress of dust, water or other contaminants via the first end 28 of the housing 12. Additionally, the sealing cap 16 can be provided allowing the button 34 and mouthpiece 26 to be sealed, thereby preventing ingress of contaminants via at the second end 30 of the housing 12. In this arrangement, the sealing cap 16 can also be adapted to lock or otherwise prevent actuation of the button 34, thereby preventing accidental activation of the inhaler.

It is also to be appreciated that the mouthpiece aperture 26.1 generally has a larger diameter than the duct aperture 36.1 to allow for medicament expelled from the canister 22 before complete alignment of the apertures 26.1 and 36.1 occurs to be inhaled. This difference in diameters between the apertures 26.1 and 36.1 accommodates for variation in movement when the duct is displaced down the conduit 32 when the button 34 is pressed.

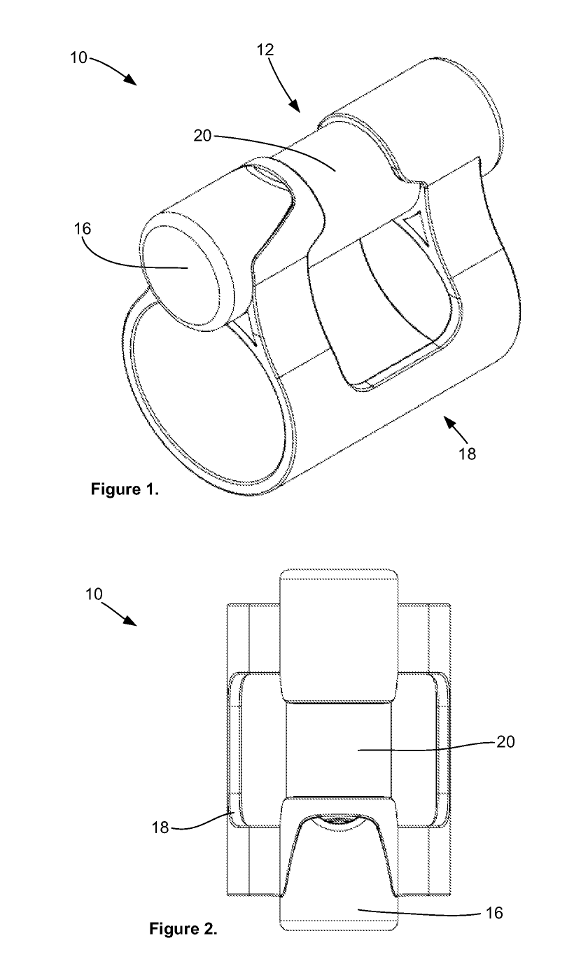

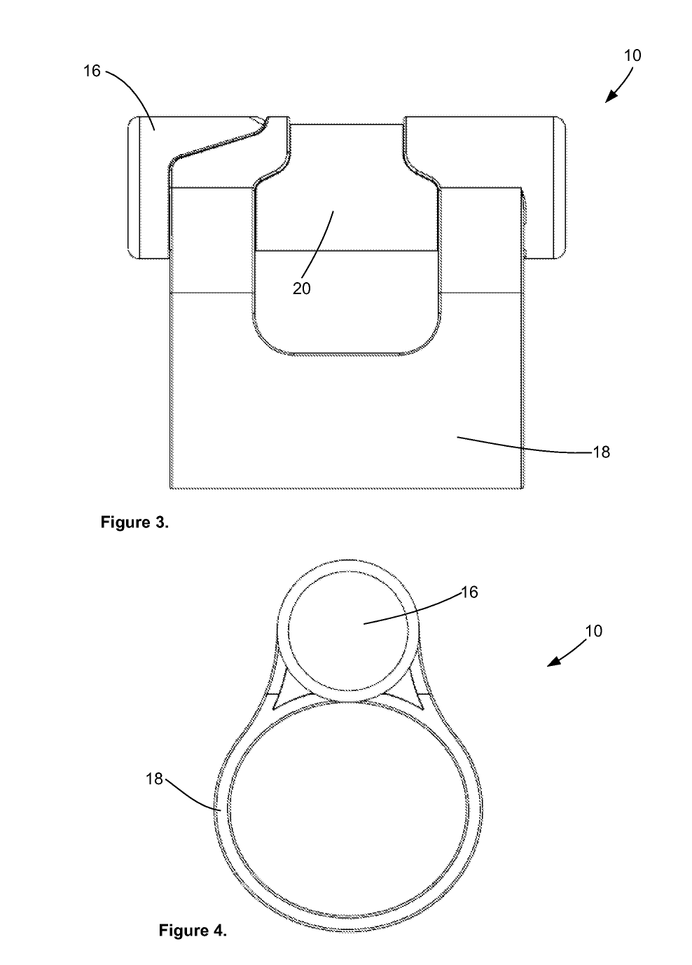

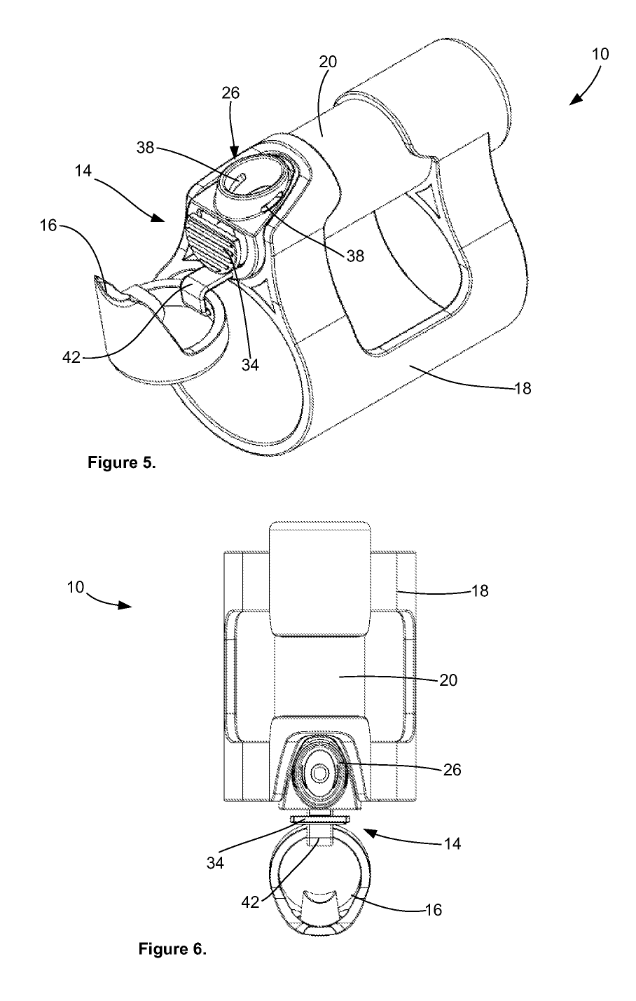

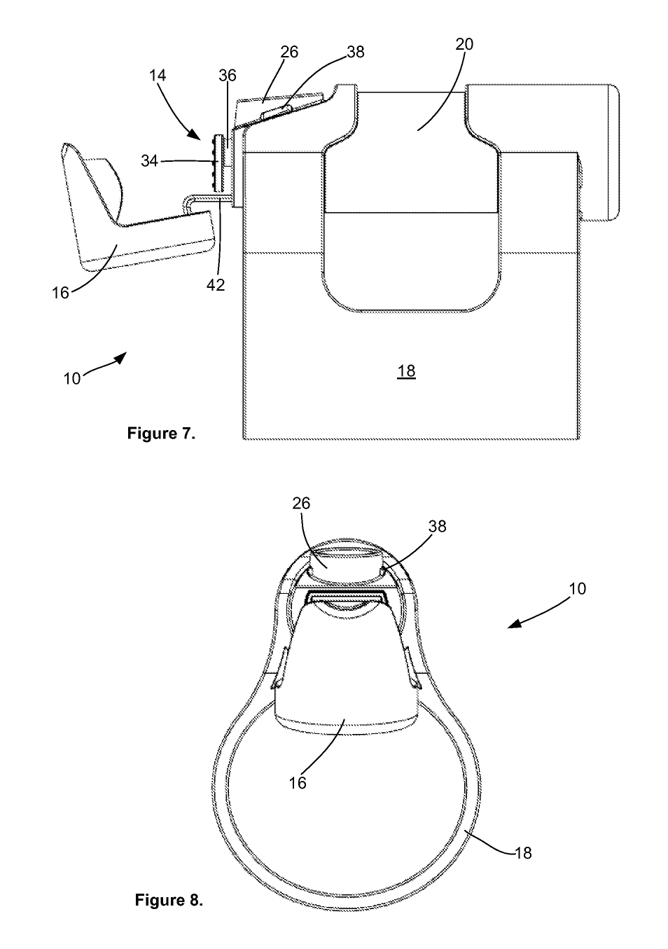

To ensure that the duct 36 and mouthpiece 26 are sealed in a watertight manner, the inhaler 10 also includes a sealing cap 16 displaceable between a closed position, in which the sealing cap 16 engages the housing 12 to cover the button 34 and the mouthpiece 26 with its air intake apertures 38, and an open position, in which the sealing cap 16 is disengaged from the housing 12 to expose the mouthpiece 26 and the button 34. FIGS. 1-4 and 10 show the sealing cap 16 in the closed position, with FIGS. 5-8 and 11 showing the sealing cap 16 in the open position, respectively.

In the current example, the sealing cap 16 engages the second end 30 of the housing 12 when in the closed position. The sealing cap 12 is configured to seal and/or lock the button 34 and seal the mouthpiece 26 in a watertight manner when in the closed position. The sealing cap 16 also covers and seals the air intake apertures 38 of the mouthpiece when in the closed position. The sealing cap 16 also typically includes a tether 42 to secure the sealing cap 16 to the housing 12 when in the open position.

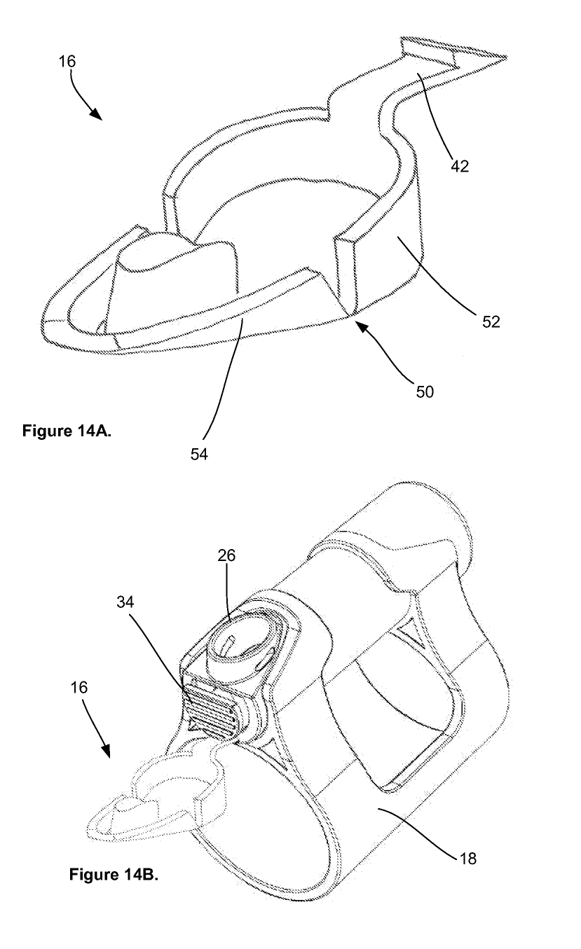

FIGS. 14A and 14B show a further example of a sealing cap 16 for the inhaler 10. In this example, the sealing cap 16 defines a hinge 50, as shown, which splits the cap into a rear portion 52 and a front portion 54. This allows the cap 16 to fold out when in the open position, allowing easier access to the button 34, i.e. more room for a user to use his/her finger to press button 34. It is to be appreciated that the hinge 50 would also be configured to establish a fluid tight seal when in the closed position. In addition, the tether 42 includes a bevelled end to assist in securing the tether to the housing 12 during manufacture. Typically, the sealing cap 16 will also define a slit to facilitate a user in opening the cap, i.e. a slit in which a user can insert a fingernail, or the like.

It is to be appreciated that the inhaler 10 finds particular application in the administration of asthma medication to people active in an aquatic environment. Particularly, the inhaler 10 is useful for surfers suffering from asthma to treat asthma attacks when in the water. However, the inhaler 10 can also be used by swimmers or other athletes active in environments where conventional inhalers might be damaged due to the ingress of dirt or liquid.

It is also to be appreciated that the attachment mechanism 18 of the inhaler 10 allows the medicament canister 22 to be sealed inside the receptacle 20 such that the canister 22 does not require displacement when actuated, as is the case with prior art inhalers. The current inhaler 10 does not require any complex or expensive to manufacture components to urge the canister towards an actuation valve. In contrast, the inhaler of the current arrangement features an actuator 14 able to actuate the canister's nozzle 24 without requiring movement of the canister 22 itself. In addition, as the actuator 14 is incorporated with and proximate to the mouthpiece 26, both the mouthpiece 26 and button 34 can be covered by a single and common sealing cap 16, making sealing of these moving parts more elegant and straightforward. This is in contrast to the traditional arrangement in which a canister activation point, typically being the bottom of the canister, is located away from the mouthpiece.

A further benefit of the current arrangement is that a user need only open a single sealing cap 16 to activate the button 34, making the inhaler 10 easier to operate specifically in an aquatic environment, such as in surf conditions, allowing it to be used even if the conditions are rough. In addition, the actuator 14 of the current arrangement does not effect the nozzle 24 and related delivery mechanism of the canister 22, so that a standard canister can be used whilst still allowing a controlled dosage of the medicament to be delivered via the inhaler 10 in accordance with normal operation of the canister.

Many modifications or variations will be apparent to those skilled in the art without departing from the scope of the present invention. All such variations and modifications should be considered to fall within the spirit and scope of the invention broadly appearing and described in more detail herein.

It is to be appreciated that reference to “one example” or “an example” of the invention is not made in an exclusive sense. Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise.

Features that are common to the art are not explained in any detail as they are deemed to be easily understood by the skilled person. Similarly, throughout this specification, the term “comprising” and its grammatical equivalents shall be taken to have an inclusive meaning, unless the context of use clearly indicates otherwise.

What is claimed is:

1. An inhaler having:
a housing which defines a receptacle shaped and dimensioned for receiving a pressurised medicament canister having a nozzle, and a mouthpiece arranged in fluid communication with the receptacle by means of a conduit; and,
an actuator including a button fast with a duct which is shaped and configured for fitment into the conduit, the duct configured to arrange the mouthpiece and nozzle in fluid communication when the button is pressed, thereby actuating the canister.

2. The inhaler of claim 1, wherein the inhaler includes a sealing cap displaceable between a closed position, in which the cap engages the housing to at least one of seal and lock the button and seal the mouthpiece, and an open position, in which the cap is disengaged from the housing to expose the mouthpiece and the button.

3. The inhaler of claim 1, wherein the inhaler includes an attachment mechanism shaped and configured for removably attaching the housing to a body of a person.

4. The inhaler of claim 1, wherein the housing includes an elongate tubular structure with a first end forming an entry to the receptacle with the mouthpiece defined on a side of the housing proximate a second end.

5. The inhaler of claim 4, wherein the housing defines the conduit substantially coaxial with the elongate tubular housing so that said conduit extends from the receptacle to the second end of the housing.

6. The inhaler of claim 4, wherein the duct extends transversely from the button so that the button lies substantially coplanar with the second end of the housing when the duct is fitted inside the conduit.

7. The inhaler of claim 6, wherein a second end of the housing is shaped and configured to receive the button so that said button lies flush with the second end of the housing.

8. The inhaler of claim 2, wherein the sealing cap engages the second end of the housing when in the closed position.

9. The inhaler of claim 3, wherein the attachment mechanism includes a receptacle cap configured to engage the first end of the housing to seal the canister inside the receptacle in a fluid tight manner.

10. The inhaler of claim 1, wherein the duct includes at least one O-ring seal to seal the duct inside the conduit in a fluid tight manner.

11. The inhaler of claim 1, wherein the duct is configured to actuate the nozzle of the canister when the button is pressed to provide a passageway for the medicament expelled from the nozzle through the duct to the mouthpiece.

12. The inhaler of claim 2, wherein the sealing cap is configured to seal the button and mouthpiece in a fluid tight manner when in the closed position.

13. The inhaler of claim 1, wherein the mouthpiece includes air intake apertures to facilitate a person operatively inhaling the medicament.

14. The inhaler of claim 13, wherein a sealing cap covers and seals the air inlet apertures when in the closed position.

15. The inhaler of claim 2, wherein the sealing cap includes a tether to secure the sealing cap to the housing when in the open position.

16. The inhaler of claim 2, wherein the sealing cap defines a hinge portion which enables the cap to split into a front portion and a rear portion in the open position.

17. The inhaler of claim 3, wherein the attachment mechanism includes an elastic strap for attaching the housing to a limb of the person's body.

18. The inhaler of claim 17, wherein the strap is configured to attach the housing to a forearm of the person such that the housing lies substantially parallel with the person's forearm.

19. The inhaler of claim 3, wherein the attachment mechanism operatively holds the canister immobile inside the receptacle.

20. The inhaler of claim 3, wherein the attachment mechanism is configured to operatively seal the canister inside the receptacle.

21. The inhaler of claim 1, which includes a pressurised medicament canister.

22. The inhaler of claim 1, wherein the mouthpiece defines a mouthpiece aperture via which the duct arranges the mouthpiece in fluid communication with the nozzle when the button is pressed.

23. The inhaler of claim 22, wherein the mouthpiece aperture defines a larger diameter than an aperture in which the duct terminates.
